(19) United States
(12) Patent Application Publication
(10) Pub. No.: US 2005/0037983 A1
(43) Pub. Date: Feb. 17, 2005

(54) Compositions and methods for the treatment of depression and other affective disorders

(76) Inventors: Timothy Dinan, Cork (IE); Peter Daly, Dublin (IE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 10/797,146

(22) Filed: Mar. 11, 2004

Related U.S. Application Data
(60) Provisional application No. 60/453,786, filed on Mar. 11, 2003.
(60) Provisional application No. 60/459,073, filed on Mar. 31, 2003.

(51) Int. Cl. A61K 31/7048; A61K 31/60; A61K 31/405; A61K 31/192; A61K 31/225

(52) U.S. Cl. 514/28; 514/570; 514/423; 514/460; 514/548; 514/165; 514/406; 514/420

(57) ABSTRACT

The present invention relates to compositions and methods for treating depression. The compositions and methods comprise the use of anti-inflammatory compounds alone or in combination with antidepressant compounds to down regulate HPA activation through the targeting of peripheral (non-CNS) cytokines.

### RELATED APPLICATIONS

This application claims the benefit of U.S. Provisional Application Ser. No. 60/453,786, filed Mar. 11, 2003 and the benefit of U.S. Provisional Application Ser. No. 60/459,073, filed Mar. 31, 2003, the contents of which are hereby incorporated by reference in their entirety.

### FIELD OF THE INVENTION

The present invention relates to compositions and methods for treating depression and other affective disorders.

### BACKGROUND OF THE INVENTION

Mood disorders are common in the United States and internationally. Approximately 18.8 million American adults, or about 9.5% of the U.S. population age 18 and older, have a mood disorder. Mood disorders include major depression, dysthymic disorder and bipolar disorder.

Major depression is characterized by feelings of intense sadness and despair, mental slowing and loss of concentration, pessimistic worry, agitation, and self-deprecation. Physical changes also occur, especially in severe or “melancholic” depression. These include insomnia or hypersomnia, anorexia and weight loss (or sometimes overeating), decreased energy and libido, and disruption of normal circadian rhythms of activity, body temperature, and many endocrine functions.

Disturbances in the hypothalamic-pituitary-adrenal axis (HPA) function are the most consistently demonstrated neuroendocrine abnormalities in major depression. Hypercortisolism and the relative failure to suppress with an overnight dose of dexamethasone are frequently seen. Elevated cerebrospinal fluid (CSF) levels of corticotropin-releasing hormone (CRH)-immunoreactivity, together with blunted CRH-stimulated adrenocorticotropic hormone (ACTH) release, have also been reported. These changes occur in a setting of adrenal hyperplasia, demonstrable either by CT or MRI imaging (Dinan, TG. Brit J Psychiat. 1984; 21: 813-829)

Depression therapy is based at present around the monoamine hypothesis, i.e. that imbalances in serotonin or noradrenaline or other neurotransmitters play a major role in the development of the disease and that correcting these such as through use of selective serotonin re-uptake inhibitors (SSRI) provides effective therapy. While such drug classes as SSRI and serotonin-norepinephrine reuptake inhibitors (SNRI) are effective it is recognised that such strategies do not fully address the underlying mechanisms and there is a significant amount of unmet clinical need. In particular only an estimated 70% of subjects respond to SSRI or SNRI therapy and of those that do a major disadvantage is the time lag of about 2 to 6 weeks before such drug begin to act.

The role of the hypothalmus-pituitary-adrenal axis in the aetiology and progression of depression has become increasingly recognized in recent years. An appreciation of the limited role for further fundamental innovation in the area of monoamine based drugs is behind recent attempts to develop drugs which impact on the neuorhormonal system such as corticotropin releasing factor (CRF) antagonists or vasopressin (V1b) antagonists. Such strategies are directed at antagonising the neurohormonal signalling between the pituitary and the adrenal cortex, which is responsible for producing cortisol.

To date all approved drugs and many investigational drugs are targeted at the brain either in terms of specific neurotransmitters or combinations of neurotransmitters or at the neurohormonal system in the brain.

There remains a need to identify and develop additional methods that can be used in the treatment of depression and other affective disorders.

### SUMMARY OF THE INVENTION

The present invention provides a method for the treatment or alleviation of depression or other affective disorders comprising administering an amount of an anti-inflammatory agent effective to treat or alleviate depression or other affective disorder to a subject in need thereof. Surprisingly, it has been found that the down-regulation of peripheral (non-CNS) cytokine levels provides for treatment or alleviation of depression or other affective disorders. Without intending to be limited by theory, it is believed that the anti-inflammatory agent acts peripherally to modulate the hypothalamic-pituitary-adrenal (HPA) axis to treat or alleviate depression or other affective disorders.

The present invention further provides a method for the treatment of depression or other affective disorder comprising administering an effective amount of an anti-inflammatory agent to a subject in need thereof, where the anti-inflammatory agent down-regulates peripheral serum levels of a pro-inflammatory molecule or up-regulates peripheral serum levels of an anti-inflammatory molecule or both.

The present invention also provides a method for potentiating the action of an antidepressant agent comprising administering an effective amount of a combination of agents to a subject in need thereof, where the combination comprises an effective amount an antidepressant agent and an amount of an anti-inflammatory agent directed against peripheral cytokines effective to treat or alleviate depression or other affective disorder.

The present invention also provides a method for the treatment or prevention of drug induced depression comprising administering an amount of an anti-inflammatory agent effective to treat or alleviate depression to a subject in need thereof.

The present invention further provides a method for the identification of an anti-inflammatory agent for use in the treatment of depression and affective disorders which comprises: (a) inducing pro-inflammatory cytokines in a test animal; (b) administering a test agent to the test animal; (c) obtaining a blood sample from the test animal; (d) assaying the blood sample; (e) determining the levels of IL-1, IL-6 and TNF in said blood; and (f) identifying a compound that down regulates pro-inflammatory cytokine production.

### DETAILED DESCRIPTION OF THE INVENTION

The present invention relates to methods for the treatment or alleviation of depression and affective disorders (e.g. mood disorders) employing the strategy of targeting peripheral (i.e. non-CNS) cytokines through administration of an effective amount of an anti-inflammatory agent to treat or alleviate depression or other affective disorders. In a preferred embodiment, the methods include the administration of anti-inflammatory agents targeted systemically to the immune system so as to modulate hypothalamic-pituitary-adrenal axis (HPA) activation to treat or alleviate depression and affective disorders. Preferably the agent employed will produce suppression of inflammatory cytokines, particularly those that activate the HPA.

The section headings are used herein for organizational purposes only, and are not to be construed as in any way limiting the subject matter described.

Definitions

As used herein, “depression and/or other affective disorders” include any disorder in which the primary symptom is a disturbance in mood (e.g. a mood disorder). Depression includes, but is not limited to, major depressive disorder, dysthymic disorder, bipolar I disorder, bipolar II disorder, and cyclothymic disorder. Other affective disorders include those such as seasonal affective disorder.

As used herein the term “anti-inflammatory agent” means those agent classes whose main mode of action and use is in the area of treating inflammation and also any other agent from another therapeutic class that possesses useful anti-inflammatory effects. Such anti-inflammatory agents include, but are not limited to non-steroidal anti-inflammatory drugs (NSAIDs), disease modifying anti-rheumatic drugs (DMARDs), macrolide antibiotics and statins. Preferably, the NSAIDs include, but are not limited to, salicylates (e.g. aspirin), arylpropionic acids (e.g. ibuprofen), anthranilic acids (e.g. mefenamic acid), pyrazoles (e.g. phenylbutazone), cyclic acetic acids (indomethicin) and oxicams (e.g. piroxicam). Preferably, anti-inflammatory agents for use in the methods of the present invention include sulindac, diclofenac, tenoxicam, ketorolac, naproxen, nabumetone, diflunasal, ketoprofen, arlypropionic acids, tenidap, hydroxychloroquine, sulfasalazine, celecoxib, rofecoxib, meloxicam, etoricoxib, valdecoxib, methotrexate, etanercept, infliximab, adalimumab, atorvastatin, fluvastatin, lovastatin, pravastatin, simvastatin clarithromycin, azithromycin, roxithromycin, erythromycin, ibuprofen, dexibuprofen, flurbiprofen, fenoprofen, fenbufen, benoxaprofen, dexketoprofen, tolfenamic acid, nimesulide and oxaprozin.

As used herein the term “antidepressant agent” refers to those agent classes whose main mode of action and use is in the area of treating depression and also any other agent from another therapeutic class that possesses useful antidepressant effects. Such anti-depressant agents include, but are not limited to imipramine, amitriptyline, desipramine, chloroimipramine, dibenzepin, doxepin, dosulepin, maprotilene, nortriptyline, mianserin, triipramine, trazadone, nefazadone, mirtazapine, reboxetine, tranylcypromine, moclobemide, brofaramine, paroxetine, fluoxetine, sertraline, fluvoxamine, citalopram, escitalopram, venlafaxine, duloxetine, buspirone, flibanserin, modafinil and buproprion.

As used herein, a “subject in need” refers to a subject suffering from depression or other affective disorder. Preferably, a subject in need has been diagnosed with depression or other affective disorder. A “subject in need” can be diagnosed with depression or other affective disorder using any method available for determining depression or other affective disorder symptoms. In a preferred aspect, such disorders can be diagnosed based on the Diagnostic and Statistical Manual of Mental Disorders, fourth edition (DSM-IV). Moreover, the severity of the depression or other affective disorder can be rated by any method of rating a disorder available. In a preferred aspect, the severity of the depression or other affective disorder can be rated using the Hamilton rating scale for depression (HAMD). Hamilton M. “Development of a rating scale for primary depressive illness.” Br J Soc Clin Psychol., 1967;6:278-296.

In another aspect, the subject in need of treatment or alleviation of depression or other affective disorder may or may not respond to antidepressant agents alone. In a preferred aspect, the subject in need is refractory to existing antidepressants. In addition, the subject may be suffering from melancholic depression. In a further aspect, the subject is both refractory to existing antidepressants and suffering from melancholic depression.

In another aspect, the subject in need is also suffering from a pre-existing cardiac or vascular disease, including, but not limited to, coronary artery disease angina and hypertension.

Methods of Treating Depression and Other Affective Disorders

Disturbances in the hypothalamic-pituitary-adrenal axis (HPA) function are the most consistently demonstrated neuroendocrine abnormalities in major depression. Hypercortisolism and the relative failure to suppress with an overnight dose of dexamethasone are frequently seen. Elevated CSF levels of corticotropin-releasing hormone (CRH)-immunoreactivity, together with blunted CRH-stimulated ACTH release, have also been reported. These changes occur in a setting of adrenal hyperplasia, demonstrable either by CT or MRI imaging (Dinan, TG. Brit J Psychiat. 1984; 21: 813-829).

There is also evidence in major depression of significant increases in pro-inflammatory cytokines such as interleukin-1 (IL-1) and interleukin-6 (IL-6) (Maes M, Human Psychopharmacol. 2001; 16: 95-103). Both of these cytokines activate the HPA and may play a significant role in sustaining the HPA activation seen in depression. It also appears that effective treatment of depression is accompanied by the suppression of pro-inflammatory cytokines and decreased activation of the HPA. As reviewed by Maes (Hum Psychopharmacol Clin Exp. 2001: 16: 95-103), various types of antidepressant drug such as tricyclic antidepressants, SSRI 's, SNRI's, lithium, MOA-I's suppress the acute phase response seen in depression and normalise levels of IL-6.

There is considerable cross-talk between the endocrine and immune systems. The pro-inflammatory cytokines interleukin-1 (IL1) and IL6 are the most potent activators of the HPA. IL-6 may be involved the etiology of depression based on observations of subjects undergoing treatment with the exogenous cytokine interferon alpha (IFN-α). Major depression is reported in up to 40% of subjects treated with IFN-α and is a common reason for treatment discontinuation. The most frequent indication for IFN-α treatment is chronic viral hepatitis. In hepatitis C, IFN-α has a direct antiviral and anti-proliferative effect on hepatocytes infected by the virus as well as an indirect effect via induction of other cytokines (Bonaccorso S et al. Psychiatry Res 2001; 15;105(1-2):45-55). IL-6 is significantly increased as early as four hours after a single dose of IFN-α compared with placebo. Musselman et al (Am J Psychiatry 2001 a; 158:1252-1257) have demonstrated a modest impact of the selective serotonin reuptake inhibitor (SSSR1) paroxetine in preventing depression in subjects treated with IFN-α. Those subjects who became depressed had higher levels of serum IL-6 following IFN-α compared with the non-depressed group, suggesting a role for IL-6 in the genesis of the observed depressive syndrome.

Thus, the hypothalmus-pituitary-adrenal axis may play a role in the etiology and progression of depression and affective disorders. In addition, development of more affective monoamine-based therapies appears limited. An increasing amount of research in this field is focused on the HPA axis and drugs such as CRF antagonists which act to modulate it. Other investigational drugs focus on other aspects of the neurohormonal axis of the brain.

Surprisingly, it has been found herein, that down-regulation of peripheral (non-CNS) cytokines treats or alleviates depression and other affective disorders. The present invention therefore provides novel methods for the treatment or alleviation of depression and other affective disorders through down regulating HPA activation by means of decreasing peripheral cytokines which activate it. Thus, the present invention comprises a somatic or systemic approach to antidepressant therapy.

Thus, one aspect of the present invention provides methods for the treatment or alleviation of depression and other affective disorders by administering an agent with anti-inflammatory activity to a subject in need thereof. In a preferred aspect the methods employ the use of agents targeted systemically at the immune system so as to modulate HPA activation to provide additional therapeutic benefits to antidepressant therapy. Preferably, the agent employed will produce suppression of inflammatory cytokines, particularly those known to activate the HPA.

In one aspect the invention comprises the use of an immunomodulatory agent to treat a subject suffering from depression or other affective disorder in such a manner as to reduce inflammatory cytokines. In a preferred aspect, the immunomodulatory agent is a NSAID, DMARD or other anti-rheumatic agent or other anti-inflammatory agent or combinations thereof. Such an agent may comprise a mixed Cox1/Cox 2 inhibitor or be a Cox-2 inhibitor.

In another aspect, the present invention provides a method for the treatment or alleviation of depression or other affective disorders comprising administering an amount of an anti-inflammatory agent effective to down-regulate peripheral serum levels of pro-inflammatory molecules or up-regulate peripheral serum levels of anti-inflammatory molecules to a subject in need of such therapy. Preferably, the down-regulated molecules include IL-1, IL-6, interferon-gamma, TFN-alpha, and activators of the IL-6 receptor or a combination thereof. Preferably the up-regulated anti-inflammatory molecules include IL-10. Even more preferably administration of the anti-inflammatory results in both the down-regulation of pro-inflammatory molecules and the up-regulation of anti-inflammatory molecules.

In a further aspect, the present invention provides a method for potentiating the action of an antidepressant agent, where the method comprises the administration to a subject in need of such therapy of an effective amount of an antidepressant agent and an amount of an anti-inflammatory agent effective to potentiate the action of the antidepressant agent.

In a preferred aspect, the method comprises administering to a subject in need of such therapy an effective amount of an antidepressant agent whereby the antidepressant agent inhibits the metabolism of the anti-inflammatory agent such that the amount of the anti-inflammatory effective to treat or alleviate depression or other affective disorders is reduced. In a more preferred aspect, the method minimizes side effects of the anti-inflammatory drug. In a preferred aspect, the antidepressant is any antidepressant agent. In one preferred aspect, the antidepressant agent is fluoxetine.

In another aspect an antidepressant agent is employed which raises serum levels of the anti-inflammatory agent. Preferably, the method reduces the amount of the anti-inflammatory agent effective in treating or alleviating depression or other affective disorders. In a more preferred aspect, the antidepressant agent is fluoxetine. It is believed that antidepressant agents, such as fluoxetine, raise the serum levels of the anti-inflammatory through inhibition of Cyctochrome P450 26D.

In another aspect, the present invention also provides methods for the prevention or treatment of drug-induced depression by the administration of an effective dose of an anti-inflammatory drug to a subject in need of such therapy. In a preferred aspect, the drug-induced depression results from treatment with interferons (such as interferon-1a and interferon 1-b) or interleukins.

In one aspect, the anti-inflammatory drug is an NSAID. In one preferred aspect, the NSAID is an R-enantiomer NSAID. Preferably the R-enantiomer is R-ketoprofen, R-flurbiprofen, R-naproxen, R-tiaprofenic, R-etodolac, R-ketorolac, R-suprofen, R-carprofen, R-pirprofen, R-indoprofen, R-benoxaprofen, or R-ibuprofen. In one aspect, the anti-inflammatory is a pure R-enantiomer.

In another aspect, the NSAID comprises a mixture of an R-enantiomer and an S-enantiomer of the NSAID. In a preferred aspect, the mixture comprises a ratio of R-NSAID to S-NSAID of at least 90:10, more preferably 95:5, most preferably 99:1.

In another embodiment the invention provides for a method of treatment of subjects suffering from depression secondary to treatment with interferon-alpha. (IFN-alpha) Major depression is reported in up to 40% of subjects treated with IFN-alpha and this is a common reason for discontinuation of the treatment. The most frequent indication for IFN-alpha treatment is chronic viral hepatitis. In hepatitis C, IFN-alpha has a direct antiviral and antiproliferative effect on hepatocytes infected by the virus as well as an indirect effect via induction of other cytokines (Bonaccorso S et al. Psychiatry Res 2001; 15;105(1-2); 45-55). IL-6 is significantly increases as early as four hours after a single dose of IFN-alpha compared to placebo. Musselman et al (Am J Psychiatry 2001a: 158: 1252-1257) have demonstrated a modest impact of the selective serotonin re-uptake inhibitor (SSRI) paroxetine in preventing depression in subjects treated with IFN-alpha. Those subjects who became depressed had higher levels of serum IL-6 following IFN-alpha compared with non-depressed groups, suggesting a role for IL-6 in the genesis of the observed depressive syndrome. The invention provides a method of treating IFN-alpha induced depression in subjects suffering from hepatitis C by using a combination of a drug which reduces serum levels of cytokines including a NSAID, DMARD or other anti-inflammatory drug in combination with a standard antidepressant drug. The invention also provides a prophylactic combination of agents which may be used to prevent the onset of depression in subjects suffering from hepatitis C who are receiving therapy using interferon-alpha.

Without being limited by theory, the invention provides an assay whereby potential anti-inflammatory agents may be screened to ascertain their utility for the treatment of depression through the mechanisms described herein. Test agents are initially screened in an appropriate animal, such as a rodent, or animal model such as the maternal deprivation model, as follows: a number of groups of test animals are each administered a test agent and levels of inflammatory cytokines and anti-inflammatory mediators are assayed from blood. Positive test agents are then selected and tested in human subjects. Test agents are administered at appropriate doses to a small number of human subjects who are subjects with depressive symptoms. Subjects are diagnosed under DSM-IV and rated according to the Hamilton scale. Levels of IL-6, IL-1, TNF-alpha and IL-10 and serum cortisol are measured before and after treatment by standard analytical techniques known in the art. Agents demonstrating the optimum suppression of pro-inflammatoy cytokines, elevation of anti-inflammatory cytokines, de-activation of HPA axis as shown by down-regulation of cortisol and optimum corresponding effect on depressive symptoms as scored by the Hamilton scale are then selected for further investigation.

In one aspect, the method comprises the steps of: (a) inducing pro-inflammatory cytokines in a test animal; (b) administering a test agent to the test animal; (c) obtaining a blood sample from the test animal; (d) assaying the blood sample; (e) determining the levels of IL-1, IL-6 and TNF in said blood; and (f) identifying a compound that down regulates pro-inflammatory cytokine production. In one aspect, the method further comprises (g) selecting from this group of candidate agents based on human toelrability. In a preferred aspect, the pro-inflammatory cytokines are induced by injecting the test animals with LPS. In one especially preferred aspect, the pro-inflammatory cytokine is IL-6. In another aspect, the test animal is a rodent, such as a mouse or rat.

Preferably, the test animal is a rodent, such as a mouse or rat.

In another aspect, the present invention provides a composition for use in the methods of the present invention for the treatment of depression or other affective disorder. Preferably, the composition comprises the combination of an antidepressant agent and an anti-inflammatory agent. In a more preferred aspect, the antidepressant for use in the composition includes, but is not limited to an antidepressant agent including a tricyclic, MOAI inhibitor, SSRI, SNRI, substance P antagonists, CRF antagonist, nefazadone or Welbutrin. The anti-inflammatory agent includes a NSAID, a DMARD or other anti-inflammatory agent. In a preferred aspect, the anti-inflammatory agent potentiates the action of the antidepressant drug.

The invention provides for the combination agents referred to above to be formulated in a single pharmaceutical entity using standard procedures and excipients known in the art. The invention further provides for a patient pack in which each component is provided separately (for instance in a blister pack) along with an information leaflet on how to use.

Preferably the invention comprises use of or combination anti-inflammatory drugs which are well tolerated and have low GI side effects. This includes the class of Cox2 NSAID drugs. In relation to mixed Cox1/Cox2 drugs this includes drugs such as ibuprofen. Additionally drug combinations which have market authorisations and which contain a NSAID in combination with a proton pump inhibitor or similar GI protective drug may be employed for the purposes described herein. This includes the combination of naproxen and lansoprazole.

An alternative approach to the problem of the GI side effects of NSAIDs is to utilise a enantiomer of a NSAID which has less gastric toxicity but which still has sufficient or preferably more anti-inflammatory effect than the racemate. It is known that R-isomers of arylpropionic acids are 100-1000 times less potent on cyclooxygenase inhibition in vitro than the S-isomers and are assumed to contribute only marginally to anti-inflammatory action. (Brune K et al. Pure enantiomers of 2-arylpropionic acids; tools in pain research and improved drugs in Rheumatology. J. Clin Pharmacol 1992. 32:944.). However a number of recent papers have shown that R-NSAIDs do have potent anti-inflammatory and analgesic action. R-flurbiprofen. does not inhibit cyclooxygenase activity, it does have additional anti-inflammatory properties mediated through NF-kappaB and does not cause gastrointestinal mucosal damage. (Tegeder, I et al. Inhibition of NF-kappaB and AP-1 activation by R- and S-flurbiprofen. FASEB J 2001 January 15: 2-4).

The use of R-NSAIDs is known in the art in relation to other diseases. For instance U.S. Pat. No. 5,955,504 discloses use of such compositions for treating colorectal cancer and U.S. Pat. No. 6,160,018 discloses use for treating Alzheimer's disease. Data presented in U.S. Pat. No. 6,160,018 in animal models shows the a number of the R-enantiomers were much less ulcerogenic than the S-enantiomers. No previous work to date has looked at use of R-NSAIDs or other enantiomers of NSAIDs in depression.

R- and S-isomers or arylpropionic acids have been shown to differentially regulate cytokine production in vitro (Mascagni, Pet al. R- and S-isomers of nonsteroidal anti-inflammatory drugs differentially regulate cytokine production. Eur. Cytokine Netw. Vol 11 No 2 Jun. 2000: 185-92). The authors showed that R-enantiomers of ketoprofen, ibuprofen and flurbiprofen are significantly more potent than the S-enantiomer in down-regulating IL-6 levels. Given the role of IL-6 in activation of the HPA axis this is of particular interest. Accordingly without being bound to any particular theory of method of action we consider that the R-enantiomers of NSAID are suitable drugs for the purposes of the anti-cytokine therapy described in this invention. We also consider that a particular S-enantiomer Dexibuprofen (S-ibuprofen) may also be a suitable drug for this purpose based on clinical experience to date.

Certain drugs with anti-rheumatic drugs mediate part of their effect by attenuating levels of inflammatory cell types through the process of apoptosis. It is known in the literature that the anti-rheumatic sulfasalazine is capable of inducing apoptosis in neutrophils. In one embodiment of this invention drugs such as sulfasalazine are employed for the purpose of treating depression through attenuation of cytokine signalling from neutrophils via this mechanism.

While NSAIDs and other drugs are employed in the art as anti-inflammatory agents other drugs which have various other main mechanisms of action may also possess anti-inflammatory effects. Such drugs may also be employed for the purposes described herein. In particular the statin class of drugs has potential use in inflammatory diseases. (March, F, Statins as novel immunomodulators; from cell to potential clinical benefit. Thrombob Haemostat 2003, October 90: 607-10). A recent study on long-term use of statins in subjects with coronary artery disease compared to subjects not using statins found that statin use was associated with lower risk of depression and anxiety. (Young-Xu, Y et al., Long term statin use and psychological well being. J Am Coll Cardiol 2003 August 42: 690-7).

Another class of drugs with anti-inflammatory effects are the macrolide antibiotics (Labro, MT. Anti-inflammatory activity of macrolides: a new therapeutic potential? J. Antimicrob Chemother 1998 March 41 SupplB:37-46). While exhibiting potent anti-bacterial activity their usefulness in non-bacterial lung conditions is believed to be mediated in part through their anti-inflammatory action. It is proposed here that such drugs as clarithromycin, azithromycin, roxithromycin and erythromycin have use as short term adjuncts to anti-depressant therapy either as an add-on to an existing antidepressant therapy programme or in combination products comprising an antidepressant combined with a macrolide drug. Additionally the first drug in the related class of ketolides, namely telithromycin is also included herein.

The use of low dose NSAID for the prevention of ischaemic events is well established in cardiology therapy and prophylaxis. It is also known that there is a connection between myocardial infarct in subjects and those who have long-term depression. It is apparent from the foregoing that the use of a NSAID or other anti-inflammatory drug as described herein will have particular benefits in subjects in which a co-morbidity exists of depression combined with a cardiac condition.

The compositions of the present invention can be used in the preparation of a medicament for the treatment of depression or other affective disorder.

Pharmaceutical Compositions

While it is possible for the compounds of the present invention to be administered neat, it may be preferable to formulate the agents as pharmaceutical compositions. As such, in yet another aspect of the invention, pharmaceutical compositions useful in the treatment or alleviation of depression or other affective disorders are provided. The pharmaceutical compositions of the invention may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

More particularly, the pharmaceutical compositions of the invention comprise an amount of at least one anti-inflammatory agent effective to treat or alleviate depression or other affective disorder, together with one or more pharmaceutically acceptable excipients. Optionally, the pharmaceutical compositions of the invention may comprise a combination of antidepressant and anti-inflammatory agents, or may include a second active ingredient useful in the treatment or alleviation of depression or other affective disorder.

Formulations of the present invention, e.g., for parenteral or oral administration, are most typically solids, liquid solutions, emulsions or suspensions, while inhaleable formulations for pulmonary administration are generally liquids or powders, with powder formulations being generally preferred. Alternative pharmaceutical compositions of the invention may be formulated as syrups, creams, ointments, tablets, and the like.

The term “pharmaceutically acceptable excipient” refers to an excipient for administration of a pharmaceutical agent, such as an anti-inflammatory agent. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions of the invention may be formulated as suspensions comprising an anti-inflammatory agent in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions of the invention may be in the form of a sterile injectable preparation, such as a sterile injectable aqueous emulsion or oleaginous suspension. This emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propane-diol. The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids such as oleic acid may likewise be used in the preparation of injectables.

Combination Therapy

It is also possible to combine any anti-inflammatory agent for use in the methods of the present invention with one or more other active ingredients useful in the treatment or alleviation of depression or other affective disorder, including compounds, in a unitary dosage form, or in separate dosage forms intended for simultaneous or sequential administration to a subject in need of treatment. When administered sequentially, the combination may be administered in two or more administrations. In an alternative embodiment, it is possible to administer one or more compounds of the present invention and one or more additional active ingredients by different routes.

The skilled artisan will recognize that a variety of active ingredients may be administered in combination with the compounds of the present invention that may act to augment or synergistically enhance the treatment or alleviation of depression or other affective disorder. Surprisingly, it has been found that the combination of an anti-inflammatory agent and an antidepressant act synergistically to treat or alleviate depression and other affective disorders.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

### Example 1

A 56 year old male subject with a 4-5 month history of depression was used in this example. The subject's complaints included low mood, anhedonia, anxiety, poor concentration, significant initial insomnia, mild anorexia and weight loss of approximately 2 kilos. Symptoms were precipitated by problems both in the work and home environment. A diagnosis of major depression was made. On the 17 item Hamilton rating scale for depression (HAMD) he had a score of 22.

He had been treated initially in primary care with citalopram 20 mg daily. After 5 weeks and no response this was switched to venlafaxine 75 mg daily. Over three weeks this was increased to 300 mg daily. No clinical improvement was seen after 4 weeks on this dose.

Rofecoxib 50 mg daily was added to the venlafaxine. After 1 week the subject reported a significant improvement and at this point his HAMD score was 11. He described the improvement as occurring within 5 days of commencing the Rofecoxib. When reviewed 2 weeks later the improvement was sustained.

### Example 2

A 67 year old female subject with a long history of recurring depression was used in this example. The current episode was present for 3 months when initially seen. She was low in mood, tearful, irritable, very anxious and had significant sleep disturbance in the form of initial and delayed insomnia. There was no clear precipitant for her symptoms. A diagnosis of major depression was made and she had a HAMD of 20.

She had been taking venlafaxine for approximately 1 year at a maximum tolerated dose of 225 mg daily. The depressive break through occurred on this dose. She was commenced on the non-steroidal anti-inflammatory Ibruprofen 400 mg three times daily, whilst remaining on the venlafaxine. She reported a symptomatic improvement by day 8 and on day 14 when she was assessed her HAMD was 8. She remained well 4 weeks later.

### Example 3

### Whole Animal Screening

The following method provides an assay for determining which drugs will be useful for the purposes of downregulating the HPA axis without the necessity of employing a animal model of depression

Rats are treated with LPS (injected i.p.) and elevation of IL-1, IL-6 and TNF are measured using specific ELISA assays. Animals suitably prepared are then injected with a test compound and the levels of the above anti-inflammatory cytokines are again assayed. While the test sample includes drugs with varying degrees of effect on the GI tract there was no way to predict that drugs with optimal effects would correspond to those with minimal GI side effects.

A small number of drugs are selected as candidates for the anti-cytokine therapy as described herein.

### Example 4

### Human Studies

Thirty six (36) subjects with DSM-IV major depression and Hamilton score greater than 20 were recruited. Subjects were randomly allocated into each of three groups
1. SSRI plus Vioxx 30 mg/day
2. Vioxx, 30 mg/day
3. Placebo
   All subjects have base line Il-6, IL-1 and cortisol levels assayed using specific assays. Biochemical assays are repeated at the end of weeks 1, 3 and 6. Highest response rates were observed in the group treated with a combination of the SSRI and Vioxx. Response rates of the Vioxx treated group were greater than that observed for placebo. Response to treatment was associated with a drop in cortisol levels and pro-inflammatory cytokines.

All publications, patents and patent applications are herein incorporated by reference to the same extent as if each individual publication, patent or patent application was specifically and individually indicated to be incorporated by reference.

1. A method for the treatment or alleviation of depression or other affective disorders comprising administering an amount of an anti-inflammatory agent effective to treat or alleviate depression or other affective disorder to a subject in need thereof.

2. The method of claim 1, wherein said anti-inflammatory agent down-regulates peripheral cytokine levels to thereby treat or alleviate depression or other affective disorder.

3. The method of claim 2, wherein said anti-inflammatory agent acts peripherally to modulate the hypothalamic-pituitary-adrenal (HPA) axis to thereby treat or alleviate depression or other affective disorder.

4. The method of claim 1 wherein said anti-inflammatory agent comprises a compound selected from the group consisting of a non-steroidal anti-inflammatory drug (NSAID), a disease modifying antirheumatic drug (DMRAD), a statin and a macrolide antibiotic.

5. The method of claim 4, wherein said NSAID is selected from the group consisting of salicylates, arylpropionic acids, anthranilic acids, pyrazoles, cyclic acetic acids oxicams and selective Cox2 inhibitors.

6. The method of claim 4 in wherein said NSAID is an R-enantiomer of said NSAID.

7. The method of claim 6 in which said R-enantiomer of said NSAID is selected from a group consisting of R-ketoprofen, R-flurbiprofen, R-naproxen, R-tiaprofenic, R-etodolac, R-ketorolac, R-suprofen, R-carprofen, R-pirprofen, R-indoprofen, R-benoxaprofen, R-ibuprofen.

8. The method of claim 6 wherein the ratio of said R-enantiomer NSAID to a S-enantiomer NSAID is at least 90:10 by weight.

9. The method of claim 8 wherein the ratio is at least 99:1 by weight.

10. The method of claim 4, wherein said anti-inflammatory agent comprises an agent selected from the group consisting of sulindac, diclofenac, tenoxicam, ketorolac, naproxen, nabumetone, diflunasal, ketoprofen, arlypropionic acids, tenidap, hydroxychloroquine, sulfasalazine, celecoxib, rofecoxib, meloxicam, etoricoxib, valdecoxib, methotrexate, etanercept, infliximab, adalimumab, or atorvastatin, fluvastatin, lovastatin, pravastatin, simvastatin clarithromycin, azithromycin, roxithromycin, erythromycin ibuprofen, dexibuprofen, flurbiprofen, fenoprofen, fenbufen, benoxaprofen, dexketoprofen, tolfenamic acid, nimesulide and oxaprozin.

11. The method of claim 1 wherein said antidepressant agent comprises an agent selected from the group consisting of imipramine, amitryptyline, desipramine, chloroimipramine, dibenzepin, doxepin, dosulepin, maprotilene, nortriptylene, mianserin, trimipramine, trazadone, nefazadone, mirtazapine, reboxetine, tranylcypromine, moclobemide, brofaramine, paroxetine, fluoxetine, sertraline, fluvoxamine, citalopram, escitalopram, venlafaxine, duloxetine, buspirone, flibanserin, buproprion and modafinil.

12. The method of claim 1, wherein said depression is selected from the group consisting of major depressive disorder, dysthymic disorder, bipolar I disorder, bipolar II disorder, cyclothymic disorder and drug-induced depression.

13. The method of claim 1 wherein said subject in need is refractory to antidepressant agents, suffering from melancholic depression or both.

14. The method of claim 1 wherein said subject in need has a pre-existing cardiac or vascular disease.

15. The method of claim 14, wherein said cardiac or vascular disease is selected from the group consisting of coronary artery disease, angina, and hypertension.

16. A method for the treatment of depression or other affective disorder comprising administering an effective amount of an anti-inflammatory agent to a subject in need thereof, wherein said anti-inflammatory agent down-regulates peripheral serum levels of a pro-inflammatory molecule or up-regulates peripheral serum levels of an anti-inflammatory molecule or both.

17. The method of claim 16, wherein said pro-inflammatory molecule is selected from the group consisting of interleukin-1, interleukin-6, interferon-gamma, TFN-alpha, and an activator of the interleukin-6 receptor.

18. The method of claim 16, wherein said anti-inflammatory molecule is interleukin-10.

19. A method for potentiating the action of an antidepressant agent comprising administering an effective amount of a combination of agents to a subject in need thereof, wherein said combination comprises an effective amount an antidepressant agent and an amount of an anti-inflammatory agent effective to treat or alleviate depression or other affective disorder.

20. The method of claim 19 wherein said antidepressant agent and said anti-inflammatory agent are formulated into a single pharmaceutical product.

21. The method of claim 19 wherein said antidepressant agent and said anti-inflammatory agent are provided in separate doses in a patient pack wherein said patient pack includes an explanatory leaflet for use by the subject.

22. The method of claim 19 in which the antidepressant agent employed is fluoxetine, whereby administration of said antidepressant agent inhibits the metabolism of the anti-inflammatory drug.

23. A method for the treatment or prevention of drug induced depression comprising administering an amount of an anti-inflammatory agent effective to treat or alleviate depression to a subject in need thereof.

24. The method of claim 23, wherein said drug-induced depression is induced by treatment with interferons or interleukins.

25. The method of claim 24, wherein said interferons are selected from the group consisting of interferon-1a and interferon 1-b.

26. The method of claim 24 wherein a combination of agents is used comprising an effective dose of an antidepressant agent and an amount of an anti-inflammatory effective in the treatment or alleviation of depression or other affective disorder.

27. The method of claim 26, wherein said antidepressant is selected from the group consisting of interferon alpha and interferon beta.

28. The method of claim 26, wherein said anti-inflammatory is selected from the group consisting of a NSAID, a DMARD, a statin and a macrolide antibiotic.

29. The method of claim 26 wherein said antidepressant and said anti-inflammatory are formulated into a single pharmaceutical composition.

30. The method of claim 26 wherein said antidepressant and said anti-inflammatory are supplied separately in a patient pack, wherein said patient pack further comprises an information leaflet for use by the subject.

31. A method for the identification of an anti-inflammatory agent for use in the treatment of depression and affective disorders which comprises:
(a) inducing pro-inflammatory cytokines in a test animal;
(b) administering a test agent to the test animal;
(c) obtaining a blood sample from the test animal;
(d) assaying the blood sample;
(e) determining the levels of IL-1, IL-6 and TNF in said blood; and
(f) identifying a compound that down regulates pro-inflammatory cytokine production.

32. The method of claim 31, further comprising the step:
(g) selecting from this group of candidate agents based on tolerability in humans.

33. The method of claim 31, wherein said test animal is a rodent.

34. The method of claim 31, wherein said inducing step comprises inducing pro-inflammatory cytokines by injecting LPS.

35. The method of claim 31, wherein said inflammatory cytokine is IL-6.
